(19) United States
(12) Patent Application Publication
(10) Pub. No.: US 2002/0087123 A1
(43) Pub. Date: Jul. 4, 2002

(54) Adhesion of heparin-containing coatings to blood-contacting surfaces of medical devices

(76) Inventors: Syed F.A. Hossainy, Fremont, CA (US); Wouter E. Roorda, Palo Alto, CA (US)

Correspondence Address: CAMERON KERRIGAN, SQUIRE, SANDERS & DEMPSEY, ONE MARITIME PLAZA, SUITE 300, SAN FRANCISCO, CA, 94111-3492, US

(21) Appl. No.: 09/753,630

(22) Filed: Jan. 2, 2001

(51) Int. Cl. A61L 2/00; A61M 5/32; A61F 2/06; A61M 29/00; B05D 3/00

(52) U.S. Cl. 604/198; 427/2.24; 606/194; 623/1.2

(57) ABSTRACT

The present invention relates to coatings of heparin derivatives on blood-contacting surfaces of medical devices, in particular on the surfaces of endoluminal stents, wherein the coatings have improved adhesion in comparison with conventional coatings heparin derivatives. Multiple component coatings are described in which compounds having therapeutic benefit are coated jointly with substances promoting adhesion. Multiple layers of coating are described including adhesion-enhancing primer layers. Surface pre-treatments enhance adhesion in some embodiments, including surface roughening. Baking of heparin-containing compounds following coating also enhances adhesion in some embodiments.

### BACKGROUND

1. Field of the Invention

The present invention relates to the field of hemocompatible coatings on medical devices and, in particular, to hemocompatible coatings including heparin and/or heparin derivatives having enhanced adhesion properties when coated on blood-contacting surfaces.

2. Description of Related Art

Continuing advances in medical technology have led to the development and use of numerous medical devices that come into contact with blood or other bodily fluids. To be concrete in our discussion, we focus herein on the particular example of medical devices coming into contact with mammalian blood, particularly human blood, not intending thereby to limit the scope of the present invention to medical devices used exclusively on human patients. In using such devices, it is important that contact of the blood or other bodily fluid with the various components of the medical device not cause therapeutically detrimental alterations to the fluid. In many cases, it is desirable to coat such devices with materials to enhance the biocompatiblity of the devices, including coatings that bioactive agents, anticoagulants, antimicrobial agents or a variety of other drugs.

It is convenient to consider blood-contacting medical devices as invasive or extra-corporal, although some devices span both classes. Invasive devices are used internally in the treatment of the patient, implanted into the patient for an indefinite or extended period of time or inserted into the patient for relatively brief periods. In many cases, the materials comprising the blood-contacting portions of the invasive device lack sufficient biocompatibility and/or hemocompatibility, tending to cause changes harmful to the patient in the blood or other fluid coming into contact with the surface (or surfaces) of the device. In such cases it is desirable to coat the surfaces of these devices with materials to enhance the biocompatiblity and/or hemocompatibility. Invasive devices that are typically coated with biocompatible or therapeutic substances include implantable artificial orthopedic devices, dental implants, intravascular catheters, emboli capturing systems, epicardial immobilization devices, grafts, stents, intraluminal prosthetic devices and artificial heart valves, among others.

There are also many examples of extra-corporal medical devices that come into contact with blood in which blood is transported and/or processed external to the patient. A few representative examples include cardiopulmonary bypass devices, kidney dialysis equipment, blood oxygenators, separators and defoaming devices, among others. Following such extra-corporal processing, the blood or other bodily fluid may be reintroduced into the patient, transported for storage and/or for introduction into another patient. In using such extra-corporal devices, it is important that contact of the blood or other bodily fluid with the various components of the device not cause therapeutically detrimental alterations to the fluid.

In some cases it is advantageous that the surface or the surfaces of the invasive or extra-corporal medical device be coated with substances having functions, wherein the coatings may serve several functions in addition to increasing the biocompatibility/hemocompatibility of the surface. Examples of such additional functions include the release of one or more therapeutic agents into the blood in appropriate dosages with appropriate timed-released characteristics and at the proper location within the patient. Thus, the medical device may serve as a convenient platform for the delivery of therapeutically beneficial drugs in addition to its other functions.

One important application related to implantable devices arises in connection with endoluminal stents, particularly as occurring in connection with percutaneous transluminal angioplasty (“PCTA”). Following balloon angioplasty, the lumen of the just-expanded vessel may contract due to several causes. An initial rebound of the walls of the vessel may occur following removal of the balloon. Further thrombosis or restenosis of the blood vessel may occur over time following the angioplasty procedure. The result is often the necessity for another angioplasty procedure or surgical by-pass. Endoluminal stents have been in use for several years in conjunction with a surgical procedure inserting a tube or stent into the vessel following the PCTA procedure to assist in retaining the desired intraluminal opening. A review of the procedure may be found in Endoluminal Stenting by Ulrich Sigwart, Ed. (W. B. Saunders, 1996). A compendium of coronary stents is given in Handbook of Coronarv Stents, 3^{rd} Ed. by P. W. Serruys and M. JB Kutryk, Eds. (Martin Dunitz Ltd., 2000). However, even with stenting, occlusions frequently recur within the stent requiring further PCTA or by-pass surgery. Such restenosis following PCTA and the insertion of a stent is sought to be prevented by the use of coated stents. Coatings on stents are often used for the delivery of anticoagulants or other medication that assist in preventing thrombosis and restenosis.

Heparin is an anticoagulant drug composed of a highly sulfated polysaccharide, the principle constituent of which is a glycosaminoglycan. In combination with a protein cofactor, heparin acts as an antithrombin (among other medical effects as described, for example, in Heparin-Binding Proteins, by H. E. Conrad (Academic Press, 1998)). Heparin is an attractive additive to coat on the surface(s) of blood-contacting devices in order to increase the hemocompatibility of the material and/or to release heparin or heparin derivatives into the blood to combat thrombosis and restenosis. For example, see the following papers appearing in Endoluminal Stenting (supra), “Heparin Stent Coatings” by Anthony C. Lunn pp. 80-83 (incorporated herein by reference), and “Efficient Endoluminal Drug Delivery for Stent Thrombosis” by Stephen R. Hanson and Nicolas A. F. Chronos, pp. 123-128 (incorporated herein by reference).

The heparin molecule contains numerous hydrophilic groups including hydroxyl, carboxyl, sulfate and sulfamino. Thus, heparin may be ionically, covalently or hydrogen bonded to reactive or hydrophilic surfaces (for example, metals), but underivatized heparin is typically difficult to coat onto hydrophobic materials. Thus, many types of derivatives of heparin with hydrophobic counter ions have been used in order to increase the ability of the heparin-counter ion complex to bind to hydrophobic surfaces. Such counter ions are typically cationic to facilitate binding with anionic heparin, and contain a hydrophobic region to facilitate bonding with the hydrophobic material. Typical heparin derivatives include, but are not limited to, heparin complex formed with typically large quaternary ammonium species such as benzylalkonium groups (typically introduced in the form of benzylalkonium chloride), tridodecylmethylammonium chloride (“TDMAC”), and the commercial heparin derivative offered by Baxter International under the tradename DURAFLO or DURAFLO II. Herein we denote as “heparin derivative,” “derivatized heparin,” or “heparin complex” any complex of heparin with a counter ion, typically a relatively large, hydrophobic counter ion. Derivatized heparin is typically only slightly soluble in aqueous or polar solutions. Examples of heparin derivatives are described in the following U.S. patents (incorporated herein by reference): U.S. Pat. Nos. 4,654,327; 4,871,357; 5,047,020; 5,069,899; 5,525,348; 5,541,167 and references cited therein.

Considerable work has been done in developing coatings for application to various medical devices in which the coatings contain at least one form of heparin or heparin derivative. Combinations of heparin and heparin derivatives with other drugs, as well as various techniques for tailoring the coating to provide desired drug-release characteristics have been studied. Examples of such work include that of Chen et. al. (incorporated herein by reference), published in J. Vascular Surgery, Vol 22, No. 3 pp. 237-247 (September 1995) and the following U.S. patents (incorporated herein by reference): U.S. Pat. Nos. 4,118,485; 4,678,468; 4,745,105; 4,745,107; 4,895,566; 5,013,717; 5,061,738; 5,135,516; 5,322,659; 5,383,927; 5,417,969; 5,441,759; 5,865,814; 5,876,433; 5,879,697; 5,993,890 as well as references cited in the foregoing patents and article.

The present invention relates to hemocompatible coatings on blood-contacting medical devices, particularly coatings containing heparin and/or derivatives of heparin. Such coatings may be used for several purposes: To increase the hemocompatibility of the surfaces upon which the coatings reside. To deliver heparin and/or heparin derivative into the blood as it contacts the coated surface. In combination with other drugs, to provide a convenient matrix from which other drugs can be delivered into the blood as the blood contacts the surface. To provide a combination of the foregoing benefits, among others, in a single coating. Thus, we can consider two general classes of benefits resulting from the use of coatings containing heparin and/or heparin derivatives: 1) increasing the biompatibility/hemocompatibility of the blood-contacting surface and, 2) the delivery of therapeutic drugs, including heparin/heparin derivatives, into the blood.

Derivatives of heparin typically contain hydrophobic counter ions referred to herein as “heparin complexes.” Such heparin complexes tend not to adhere very well to metal surfaces, tending to dissipate from the surface, leaving uncoated surface in contact with blood. Thrombosis or other detrimental effects in the blood are a possible result. The present invention relates to compositions and procedures for coating heparin complexes on medical devices, typically metallic surfaces, such that adhesion of the heparin complex to the surface is improved. Thus, heparin coatings persist for a longer period of time in contact with blood, reducing the possibilities of thrombosis, restenosis or other detrimental alterations occurring in the blood.

### SUMMARY

The present invention relates to coatings of heparin complexes on blood-contacting surfaces of medical devices, particularly endoluminal stents and most particularly stainless steel endoluminal stents. Advantages of the present invention include providing a drug delivery platform in the form of a coated medical device while retaining hemocompatibility throughout its use. Some embodiments include roughening of the surface is prior to coating, typically by means of plasma etching. Argon plasma etching of the surface is one means to roughen stainless steel surfaces. Some embodiments include the use of dip coating of heparin-containing compound onto the surface followed by high temperature baking to fix the heparin-containing compound in place. Following coating, the coating may be baked at high temperature to achieve a firm bond between the heparin-containing compound and the surface. Typical temperatures are approximately 50-60 deg. C. up to approximately 100 deg. C.

Multiple coating layers are employed in some embodiments, typically coating the upper layers such that these upper layers of have differing compositions and/or other properties. Some embodiments use a mixture, blend or other formulation of heparin-containing compound in combination with an adhesion-enhancing substance such that the heparin-containing compound becomes more tightly adhered to the surface in combination with the enhancer than heparin-containing compound does by itself. Hydrophobic as well as hydrophilic enhancers are used. A primer coating layer is used in some embodiments to enhance the adhesion of a later-applied layer. Application of the primer coating layer by means of the dip coating and baking is optionally performed. Ethylene vinyl alcohol copolymer (“EVAL”) is one example of a primer known adhere strongly to metal surfaces.

Mixtures of heparin complex (typically DURAFLO) with EVAL are described that may be applied in a single coating step and that demonstrate good heparin adhesion to stainless steel surfaces. Stainless steel coupons coated with heparin/EVAL pursuant to some embodiments of the present invention tested positive for heparin following 72 hour immersion in water.

### BRIEF DESCRIPTION OF THE FIGURES

This application has no figures.

### DETAILED DESCRIPTION

The present invention relates to coatings of heparin derivative on blood-contacting surfaces of medical devices in such manner as to enhance the adhesion of such coatings in comparison with conventional coatings of heparin derivatives. For economy of language we use “heparin-containing compound” as a generic expression to indicate without distinction a heparin derivative, utilized either singly or in combination with other forms of heparin, or in combination with one or more other substances. Specific reference will be made to the precise form of heparin-containing compound if relevant. “Heparin-containing coating” indicates a coating in which at least one component thereof is a heparin-containing compound.

To be concrete in our discussion, we describe the example of coating heparin derivatives onto endoluminal stents. As presently used, such stents are typically made of stainless steel. However, the techniques of the present invention can be used for enhanced coating of heparin-containing compounds onto other forms of metals, alloys and non-metals as such would typically find use in bloodcontacting medical devices. Other stent materials include “MP35N,” “MP20N,” elastinite (Nitinol), tantalum, nickel-titanium alloy, platinum-iridium alloy, gold, magnesium, or combinations thereof. “MP35N” and “MP20N” are trade names for alloys of cobalt, nickel, chromium and molybdenum available from standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum. The stent also may be made from bioabsorbable or biostable polymers. “Enhanced coating” as used herein indicates a favorable combination of coating properties including adhesion of the heparin-containing compound to the surface for adequate persistent hemocompatibility as well as appropriate time-release of drug(s) into the blood for therapeutic benefit.

Surface Roughening

Some embodiments of the present invention make use of preparing the surface of the medical device prior to coating in order to enhance the adhesion of the heparin-containing compound. In one method of surface preparation, the surface is roughened prior to coating. Roughening the surface increases the surface area available for bonding and may, in addition, expose more reactive surface binding sites to coating by the heparin-containing compound including reactive grain boundaries, surface dislocations and other surface atoms having less than full coordination. Additional advantages of surface roughening prior to coating may include the removal of oxide or other passivating layers on the surface of the metal that tend to prevent or hinder tight binding between the heparin-containing compound and the surface. Roughening on a scale of nanometers is found to be effective in enhancing the adhesion of heparin-containing compounds. Typical roughening techniques are plasma etching. In particular, argon plasma etching of the surface is found to be a convenient means to roughen the surface of stainless steel as used in stents.

Elevated Temperature Bake

Some embodiments of the present invention include the coating of the heparin-containing compound onto the surface of the medical device followed by baking at elevated temperature to fix the heparin-containing compound in place. Dip coating and spray coating are among the coating techniques that can be employed. It is believed that the firm bond between the heparin-containing compound and the surface is due to hydrogen bonding with the metal, but other bonding methods and forms of attraction between the metal and the heparin-containing compound are included within the scope of these embodiments. Typical temperatures are approximately 50-60 deg. C, up to approximately 100 deg. C. Temperatures in excess of the degradation temperature of heparin-containing compound are contraindicated. However, even temperatures exceeding the degradation temperature of the heparin-containing compound may be employed if the coated surface is maintained at these high temperatures for a sufficiently short period of time that only tolerable degradation of heparin-containing compound occurs. Typical bake times range from a few minutes up to approximately several hours, although the precise time of baking can be determined by simple experimentation for the particular combination of heparin-containing compound, heparin derivative, surface material, surface pre-treatment (if any), desired coating thickness, among other factors.

Multicomponent Coatings

Methods for improving the adhesion of heparin-containing compound to metal surfaces include the addition of a blood-compatible adhesion-enhancing substance to the heparin-containing compound and causing the combination to adhere to the surface. That is, a mixture, blend or other formulation of heparin-containing compound and an adhesion-enhancing substance is prepared such that the heparin-containing compound becomes more tightly adhered to the surface in combination with the enhancer than heparin-containing compound does by itself. An entrapment of the heparin-containing compound within a matrix of tightly adhering enhancer substance is one mechanism by which enhanced adhesion may be achieved, although other adhesion-enhancing methods are possible within the scope of the present invention. However, the adhesion enhancers must not be too blood-incompatible such that the hemocompatiblity of the surface of the medical device is unacceptably degraded. Typical enhancers include polyethylene glycol (“PEG”), polyethylene oxide (“PEO”), polyvinylpyrrolidone (“PVP”), polyvinyl alcohol (“PVA”), polycaprolactone (“PCL”), polyglycolic acid (“PGA”), ethylene vinyl alcohol copolymer (“EVAL”), hyaluronic acid, polyurethanes, PCL-PEG copolymers, PCL-PGA copolymers and also BIOSPAN and derivatives of BIOSPAN (a segmented polyurethane available from The Polymer Technology Group, Inc. of Berkeley, Calif.). Hydrophobic as well as hydrophilic enhancers may be used. Copolymers, including absorbable copolymers, polyurethanes, among others can also be used. Enhancers with reasonable solubility in the solution of heparin-containing compound used for coating is also a desirable property of the enhancers.

Primer Layers

A primer (or “primary”) coating layer is used herein to indicate a layer applied to the surface to be coated in order to enhance the adhesion of a later-applied layer. The primer layer may, but need not, have hemocompatibility or drug release properties itself, but its function is to enhance adhesion of subsequent layer(s) having at least one such property. Application of the primer coating layer by means of the dip coating and baking may optionally be performed.

Ethylene vinyl alcohol copolymer (“EVAL”) is known to be tenaciously adherent to metal surfaces, including such metals and alloys as typically used in medical devices. Thus, some embodiments of the present invention relate to the coating of the surface with strongly adhered EVAL prior to coating with heparin-containing compound. The EVAL primer layer may be applied by dip or spray coating or by any other convenient technique. The bond between the metal and the EVAL primer coating serves to stabilize the later-applied coating (or multiple coatings) of heparin-containing compound. Some embodiments make use of a primer layer of EVAL followed by an application of heparin-containing compound with a relatively low concentration of heparin-containing compound. Subsequent layers of heparin-containing compound in a multi-layer bonding procedure would typically be applied so as to have increasing concentrations of heparin-containing compound, resulting in relatively highly concentrated heparin-containing compound at the upper layers for release into the blood, but tightly bonding layers upon the metal surface.

Other primer layers that can be used to enhance the adhesion of hemocompatible layers include polylysine, polycysteine, reactive silanes (such as trimethoxy silanes), chlorosilanes that may optionally have a functional head. Typical functional heads may include (when present), unsaturated functionality, —NH₂—, —COOH. Functional heads may be chosen so as to further stabilize the heparin-containing compound in contact with the primer layer on the stent. Such functional heads may optionally be modified by PEG or hyaluronic acid. The functional heads thus modified may increase the hemocompatibility resulting from application of heparin-containing compound or heparin-containing compound derivatives.

Dip coating of the primers described herein tends to give more uniform coatings and improved performance over spray coating. However, spray coating is not excluded and can be used effectively in certain instances, especially relating to the application of later-applied layers in multiple coatings. Baking the primer coating or subsequent coatings may optionally be performed in those cases in which the increased adhesion resulting therefrom is more beneficial to the performance of the coating than is the degradation typically resulting from exposure of heparin-containing compound or heparin-containing compound derivatives to elevated temperatures.

### EXAMPLES

The following examples relate to a multicomponent coating comprising ethylene vinyl alcohol copolymer (“EVAL”) and the heparin complex commercially available under the tradename DURAFLO. The EVAL/DURAFLO blend was applied to stainless steel coupons in the form of a solution wherein the solvent comprises dimethyl sulfoxide (“DMSO”) and tetrahydrofuran (“THF”). Various proportions of EVAL, DURAFLO, DMSO and THF were tested as described in detail in the following examples. In all cases, a solution having the specified percentages (weight/weight) was prepared and heated at a temperature of about 55° C. on a hotplate to achieve complete dissolution. The solution was applied dropwise from a transfer pipette while warm onto a stainless steel metal coupon and smeared on the metal. The resulting thin coating on the coupon was dried in a convection oven for about 12 hours at 50° C. The following formulations were tested:

A) EVAL 151B-DURAFLO-DMSO-THF: 2.2%-2.3%-68%-27.5%.

B) EVAL 151B-DURAFLO-DMSO-THF: 2.2%-1.2%-68%-28.6%

C) EVAL 151B-DURAFLO-DMSO-THF: 2.2%-0.6%-68.5%-28.7%

Additional formulations were tested including dimethyl acetamide (“DMAC”) as follows:

D) EVAL 151B-DURAFLO-DMSO-THF-DMAC: 2.0%-2.0%-62.8%-27.6%-5.6%

E) EVAL 151B-DURAFLO-DMSO-THF-DMAC: 2.0%-1.1%-63.4%-27.8%-5.6%

“EVAL 151B” is a commercial embodiment of EVAL sold by EVALCA Company of America of Lisle, Ill. Product information and material safety data sheet for EVAL 151B are attached hereto.

Upon drying, all coupons demonstrated a good coating judged by visual inspection of the coating. No coating cracked or peeled upon physical bending of the coupon. Coated coupons were immersed in room temperature water for 72 hours, dried in ambient air and tested for heparin by means of a Toluidine Blue stain test. All of the above coupons, Examples A-E, exhibited a positive test for heparin following 72 hours of water immersion. In addition, the intensity of the stain tends to increase with increasing ratio of DURAFLO/EVAL for the above examples, indicating increased heparin retention with increased DURAFLO/EVAL ratio.

An important advantage of the above coatings is that the mixture of DURAFLO and EVAL can be applied in a single step, eliminating the need for a separate primer coating applied in a separate processing step. Thus, EVAL-DURAFLO mixtures enhance heparin adhesion while not adding processing steps.

Having described the invention in detail, those skilled in the art will appreciate that, given the present disclosure, modifications may be made to the invention without departing from the spirit of the inventive concept described herein. Therefore, it is not intended that the scope of the invention be limited to the specific embodiments illustrated and described.

We claim:

1) A method of coating a blood-contacting surface with a heparin-containing compound comprising:
a) applying a first hemocompatible coating to said surface wherein said first hemocompatible coating is sufficiently tightly bonded to said surface so as to remain on said surface in contact with blood; and,
b) applying at least one second hemocompatible coating sequentially on said first hemocompatible coating wherein said at least one second hemocompatible coating comprises one or more therapeutic heparin-containing compounds releasable into blood.

2) A method as in claim 1 wherein said first hemocompatible layer includes a heparin-containing compound.

3) A method as in claim 1 further comprising roughening said surface prior to coating.

4) A method as in claim 1 further comprising applying a primer layer to said surface prior to applying said first hemocompatible coating, wherein said primer layer enhances adhesion of said first hemocompatible coating to said surface.

5) A method as in claim 4 wherein said primer layer is selected from the group consisting of heparin-containing compounds, ethylene vinyl alcohol copolymer, polycystine, polylysine and reactive silanes including trimethoxysilanes.

6) A method as in claim 4 wherein said primer layer contains at least one chlorosilane compound.

7) A method as in claim 6 wherein said at least one chlorosilane has a functional head.

8) A method as in claim 7 wherein said functional head of said at least one chlorosilane has functionality selected from the group consisting of unsaturated functionality, amine functionality, carboxyl functionality.

9) A method as in claim 8 wherein said functionality is modified by polyethylene glycol or hyaluronic acid.

10) A method as in claim 7 wherein said at least one second hemocompatible layer comprises a plurality of layers and wherein said plurality of layers have varying properties.

11) A method as in claim 10 wherein said varying properties comprise varying compositions.

12) A material having a hemocompatible surface produced by the method of claim 1.

13) A medical device wherein at least one surface thereof contacts blood and wherein at least a portion of said blood contacting surface is the material of claim 12.

14) A medical device as in claim 13 wherein said medical device is an endoluminal stent.

15) A method of coating a blood-contacting surface with a heparin-containing compound comprising:
a) providing a formulation containing at least one heparin-containing compound and at least one adhesion enhancer; and,
b) coating said surface with said formulation.

16) A method as in claim 15 wherein said at least one adhesion enhancer is selected from the group consisting of polyethylene glycol, polyethylene oxide, polyvinylpyrrolidone, polyvinyl alcohol, polycaprolactone, polyglycolic acid, ethylene vinyl alcohol copolymer, hyaluronic acid, polyurethanes, copolymers of polycaprolactone and polyglycolic acid, copolymers of polycaprolactone and polyethylene glycol, segmented polyurethanes and mixtures thereof.

17) A method as in claim 16 wherein said coating is performed by dip coating.

18) A method as in claim 15 further comprising roughening said surface prior to coating.

19) A material having a hemocompatible surface produced by the method of claim 15.

20) A medical device wherein at least one surface thereof contacts blood and wherein at least a portion of said blood contacting surface is the material of claim 19.

21) A medical device as in claim 20 wherein said medical device is an endoluminal stent.

22) A method of coating a blood-contacting surface with a heparin-containing compound comprising:
a) roughening said surface prior to coating; and,
b) coating said surface with a heparin-containing compound; and,
c) baking said surface and said coating thereon sufficient to affix said coating to said surface.

23) A method as in claim 22 wherein said baking is at a temperature from approximately 50 degree C. to approximately 100 degree C.

24) A method as in claim 22 wherein said coating is performed by dip coating.

25) A method as in claim 22 wherein said roughening is performed by argon plasma etching.

26) A material having a hemocompatible surface produced by the method of claim 22.

27) A medical device wherein at least one surface thereof contacts blood and wherein at least a portion of said blood contacting surface is the material of claim 26.

28) A medical device as in claim 27 wherein said medical device is an endoluminal stent.

29) A heparin-containing composition for coating onto a blood-contacting surface comprising ethylene vinyl alcohol copolymer, at least one heparin complex, dimethyl sulfoxide and tetrahydrofuran.

30) A heparin-containing composition as in claim 29 further comprising dimethyl acetamide.

31) A heparin-containing composition as in claim 29 wherein said ethylene vinyl alcohol copolymer is about 2.2% by weight of said composition.

32) A heparin-containing composition as in claim 31 wherein said heparin complex is from about 0.6% by weight to about 2.3% by weight of said composition.

33) A heparin-containing composition as in claim 30 wherein said ethylene vinyl alcohol copolymer is about 2% by weight of said composition.

34) A heparin-containing composition as in claim 31 wherein said heparin-complex is from about 1.1% by weight to about 2.0% by weight of said composition.

35) A medical device wherein at least one surface thereof contacts blood and wherein at least a portion of said blood contacting surface is coated with the material of claim 29.

36) A medical device as in claim 35 wherein said medical device is an endoluminal stent.
